(19) United States
(12) Patent Application Publication
(10) Pub. No.: US 2025/0359999 A1
(43) Pub. Date: Nov. 27, 2025

(54) EXPANDABLE LATERAL LUMBAR INTERBODY SPACER

(71) Applicant: Astura Medical Inc., Irving, TX (US)

(72) Inventors: Anthony Valkoun, Irving, TX (US); Thomas Purcell, Irving, TX (US)

(73) Assignee: Astura Medical Inc., Irving, TX (US)

(21) Appl. No.: 19/218,561

(22) Filed: May 26, 2025

Related U.S. Application Data
(60) Provisional application No. 63/651,902, filed on May 24, 2024.

(51) Int. Cl. A61F 2/44; A61F 2/30

(52) U.S. Cl. CPC A61F 2/4425; A61F 2002/30153; A61F 2002/30579; A61F 2002/443

(57) ABSTRACT

An expandable lateral lumbar interbody spacer for placement between adjacent vertebrae configured to have a collapsed state suitable for insertion into an intervertebral space between a pair of adjacent vertebrae, and an expanded state that includes lordosis change during vertical expansion.

### CROSS-REFERENCE TO RELATED APPLICATIONS

This application claims the benefit of U.S. Provisional Application No. 63/651,902, titled EXPANDABLE LATERAL LUMBAR INTERBODY SPACER, filed May 24, 2024, which is incorporated herein by reference.

### FIELD

The present invention relates generally to the field of surgery, and more specifically, to an expandable lateral lumbar interbody spacer for placement in intervertebral space between adjacent vertebrae.

### BACKGROUND

A spinal disc can become damaged as a result of degeneration, dysfunction, disease and/or trauma. Conservative treatment can include non-operative treatment through exercise and/or pain relievers to deal with the pain. Operative treatment options include disc removal and replacement. In surgical treatments, interbody implants may be used between adjacent vertebra, resulting in spinal fusion of the adjacent vertebra.

A fusion is a surgical method wherein two or more vertebrae are joined together (fused) by way of interbody implants, sometimes with bone grafting, to form a single bone. The current standard of care for interbody fusion requires surgical removal of all or a portion of the intervertebral disc. After removal of the intervertebral disc, the interbody implant is implanted in the interspace.

Interbody implants must be inserted into the intervertebral space in the same dimensions as desired to occupy the intervertebral space after the disc is removed. This requires that an opening sufficient to allow the interbody implant must be created through surrounding tissue to permit the interbody implant to be inserted into the intervertebral space. In some cases, the intervertebral space may collapse prior to insertion of the interbody implant. In these cases, additional hardware may be required to increase the intervertebral space prior to insertion of the implant.

Expandable Lateral Lumbar spacers are typically limited in expansion capability at smaller footprints due to a limitation of available material, in addition they typically expand in a single plane. They implants have endplates which expand at constant rates, which does not allow the surgeon to obtain adequate lordosis with a partially expanded implant.

Some of the implants use converging ramp mechanisms to increase the height and width of the implant. The issue with converging ramps is that they can bind of there is unequal translation of the ramps.

Typical expanding implants use a proximal drive screw to expand the implant, which did not allow the delivery of graft to into the implant.

It would be desirable to insert an interbody implant into the intervertebral space at a first smaller dimension and once in place, then expand the implant including height and width expansion, with lordosis in conjunction with height expansion.

### SUMMARY

Disclosed is an expandable lateral lumbar interbody spacer (“expandable spacer”) that is configured to have a collapsed state suitable for insertion into an intervertebral space defined by a pair of adjacent vertebrae, and an expanded state that includes lordosis change during expansion.

The disclosed expandable spacer is configured to expand the proximal and distal endplates at different expansion rates that allows maximum lordosis at partial expansion. The expandable spacer uses endplates with parallel ramps so they are pulled up ramps to ensure expansion and no binding.

The disclosed expandable spacer design also moves the drive screw to the distal portion of the implant, which opens up space in the proximal portion for graft.

By inserting the expandable cage into the intervertebral space in the initial collapsed state, it is possible to perform the surgery percutaneously with minimal disruption to tissues surrounding the surgical site and intervening soft tissue structures.

### BRIEF DESCRIPTION OF THE DRAWINGS

FIG. 1 is a perspective distal view of an expandable lateral lumbar interbody spacer.

FIG. 2 is a perspective exploded view of the expandable lateral lumbar interbody spacer of FIG. 1.

FIG. 3 is a perspective proximal view of the expandable spacer with the upper endplates omitted for clarity.

FIG. 4 is a top view with the upper endplates omitted for clarity.

FIG. 5 shows vertical expansion of the upper endplates and the lower endplates engaging a combination of steep and shallow ramps on the vertical expansion wedges.

FIG. 6 is a perspective of the anterior and posterior vertical expansion wedges.

FIGS. 7A, 7B side views showing vertical expansion of the expandable spacer.

FIG. 8 shows the expandable spacer collapsing.

FIGS. 9A-9D is an end view the expandable spacer showing the unequal expansion rates of the upper and lower anterior endplates and the upper and lower posterior endplates.

### DETAILED DESCRIPTION

The present invention is directed to an expandable lateral lumbar interbody spacer (“expandable spacer”) having anterior and posterior endplates that are configured to expand vertically at different expansion rates, creating lordosis during expansion. The expandable spacer expands from a collapsed state for insertion between the adjacent vertebrae with minimal spacer dimensions, to an expanded state supporting the adjacent vertebrae. During expansion, the anterior endplates expand unequally with the posterior endplates. This is achieved by changing the rate of expansion between the anterior and posterior upper and lower endplates.

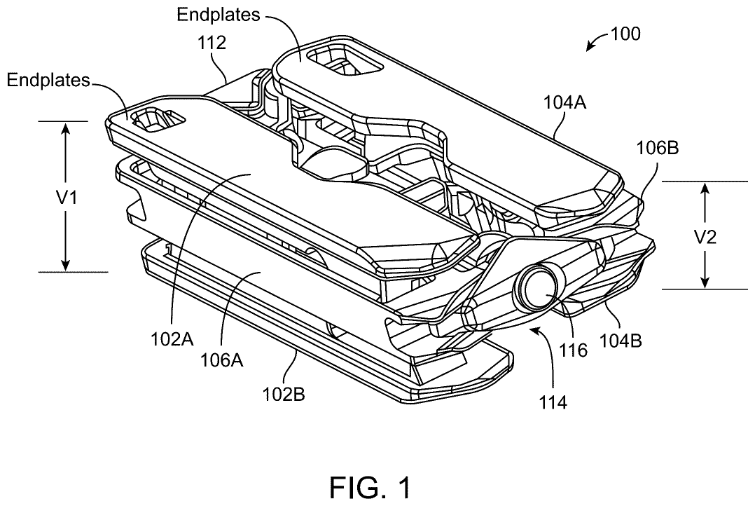

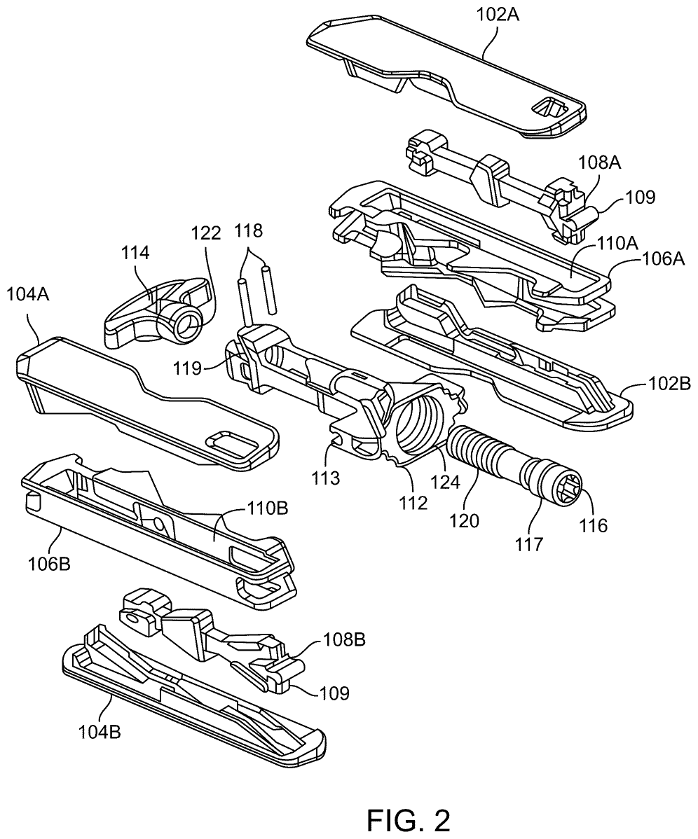

FIG. 1 is a perspective view and FIG. 2 is a perspective exploded view showing an expandable lateral lumbar interbody spacer 100 (“expandable spacer”) having 13 components: a frame, a drive housing, two lateral expansion wedges, two vertical expansion wedges, a drive screw, two upper endplates, two lower end plates, and two retention pins. The upper and lower end plates include various ramps designed to slidingly engage corresponding ramps on the vertical expansion wedges to expand the expandable lateral lumbar interbody spacer 100 (discussed in more detail below).

The expandable spacer 100 includes upper and lower anterior endplates 102A, 102B, upper and lower posterior endplates 104A, 104B, anterior and posterior lateral expansion wedges 106A, 106B, and anterior and posterior vertical expansion wedges 108A, 108B. The vertical expansion wedges 108A, 108B are positioned within slots 110A, 110B of the lateral expansion wedges 106A, 106B. In the embodiments shown, the anterior endplates 102A, 102B expand laterally L from the posterior endplates 104A, 104B at an equal rate, but the upper and lower anterior endplates 102A, 102B and the upper and lower posterior endplates 104A, 104B expand vertically at unequal rates, creating lordosis between the anterior and posterior endplates during expansion.

The expandable spacer 100 includes a drive means for expansion. In the embodiment shown, the drive means includes a drive housing 112 and a frame 114 coupled with a drive mechanism, such as a drive screw 116. The drive screw 116 is held in the drive housing 112 via a circumferential slot 117 configured to engage retention pins 118 positioned within retention pin holes 119 of the drive housing 112. The drive screw 116 includes a threaded portion 120 coupled with a threaded bore 122 in the frame 114. The drive housing 112 further includes a threaded bore 124 configured to couple with an insertion tool (not shown) for insertion of the expandable spacer 100 between adjacent vertebrae. The drive screw 116 is positioned toward the distal end of the drive housing 112, which opens up a space within the expandable spacer 100 for bone graft delivery and insertion through the threaded bore 124 of the drive housing 112.

In the embodiments shown, the upper and lower anterior endplates 102A, 102B are configured to expand laterally L away from the upper and lower posterior endplates 104A, 104B at an equal rate. The upper and lower anterior endplates 102A, 102B are configured to expand vertically V1 at a first rate, and the upper and lower posterior endplates 104A, 104B are configured to expand vertically V2 at a second rate. The first and second rates are not the same. So, the vertical expansion V1 of the upper and lower anterior endplates 102A, 102B and vertical expansion V2 of the upper and lower posterior endplates 104A, 104B are done at unequal rates, creating lordosis. Lordosis becomes greater the more the expandable spacer 100 is expanded vertically.

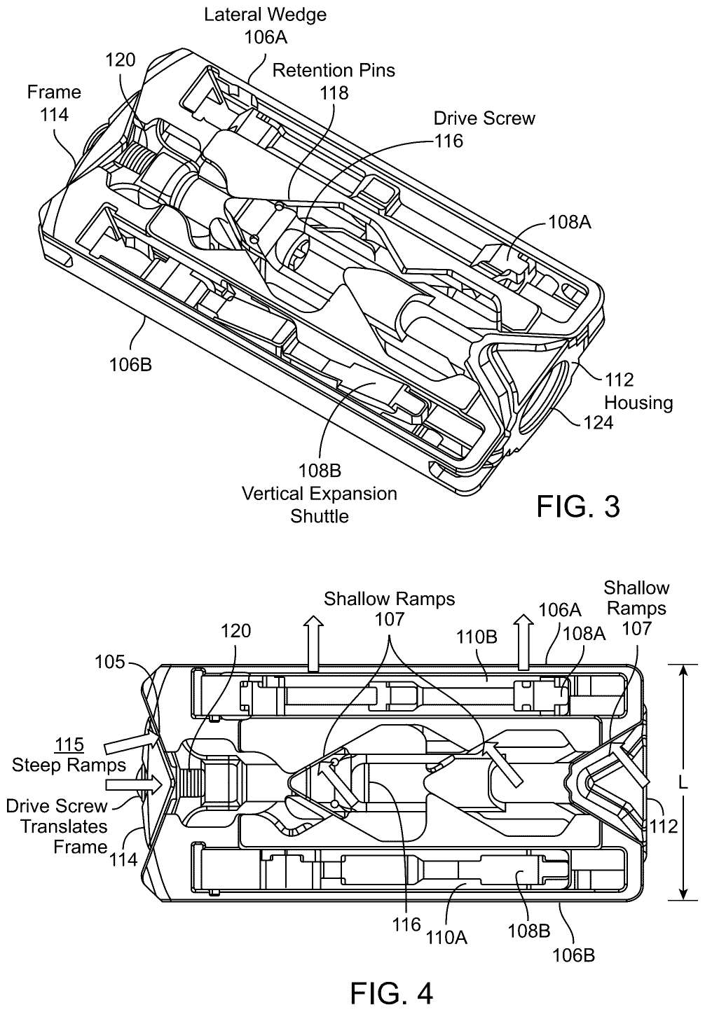

FIG. 3 is a perspective proximal view of the expandable spacer 100 with the upper endplates omitted. FIG. 4 is a top view with the upper endplates omitted for clarity. For lateral expansion, the drive screw 116 is rotated clockwise with an instrument, which translates the frame 114 toward the drive housing 112. The frame 114 includes “steep ramps” 115 which contacts corresponding “steep ramps” 105 of lateral expansion wedges 106A, 106B causing them to translate up the “shallow ramps” 107, inducing lateral expansion L of the lateral expansion wedges 106A, 106B.

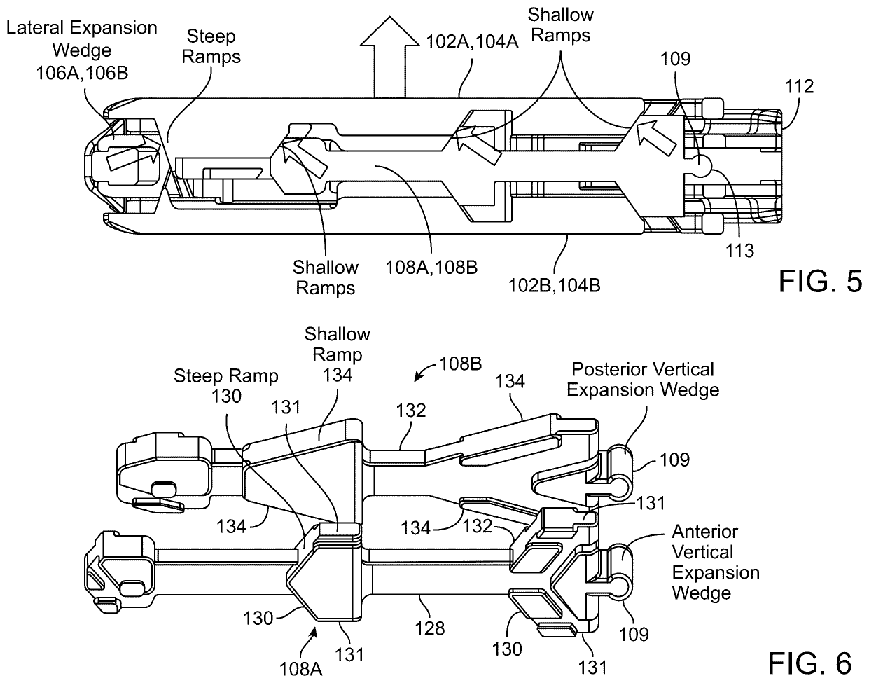

FIG. 5 shows vertical expansion of the upper endplates 102A, 104A and the lower endplates 102B, 104B engaging a combination of steep and shallow ramps on the vertical expansion wedges 108A, 108B. The expansion wedges 108A, 108B further include d-rails 109 that couple with a slot or groove 111 of the drive housing 112. The steep and shallow ramps on the vertical expansion wedges 108A, 108B are different, creating a variance in expansion between the anterior endplates 102A, 102B and the posterior endplates 104A, 104B. The anterior endplates 102A, 102B are coupled to the anterior vertical expansion wedge 108A and are configured to expand at a first vertical expansion V1 rate, and the posterior endplates 104A, 104B are coupled to the posterior vertical expansion wedge 108B and are configured to expand them at a second vertical expansion V2 rate. The difference between the first and second expansion rates create lordosis during expansion.

FIG. 6 is a perspective of the anterior and posterior vertical expansion wedges 108A, 108B. Unequal anterior endplate/posterior endplate expansion (lordosis) is achieved by changing the rate of expansion between the anterior endplates 102A, 102B and posterior endplates 104A, 104B. This is achieved by creating variance between the angle of the anterior and posterior ramp angles.

The anterior vertical expansion wedge 108A includes a wedge body 128 with multiple steep ramps 130 with flat sections 131. The posterior vertical expansion wedge 108B includes a wedge body 132 with multiple shallow ramps 134. During expansion, the vertical expansion wedges 108A, 108B translate together, and the steep ramps 130 engaging the upper and lower anterior endplates 102A, 102B and the shallow ramps 132 engage and vertically translate the upper and lower posterior endplates 104A, 104B.

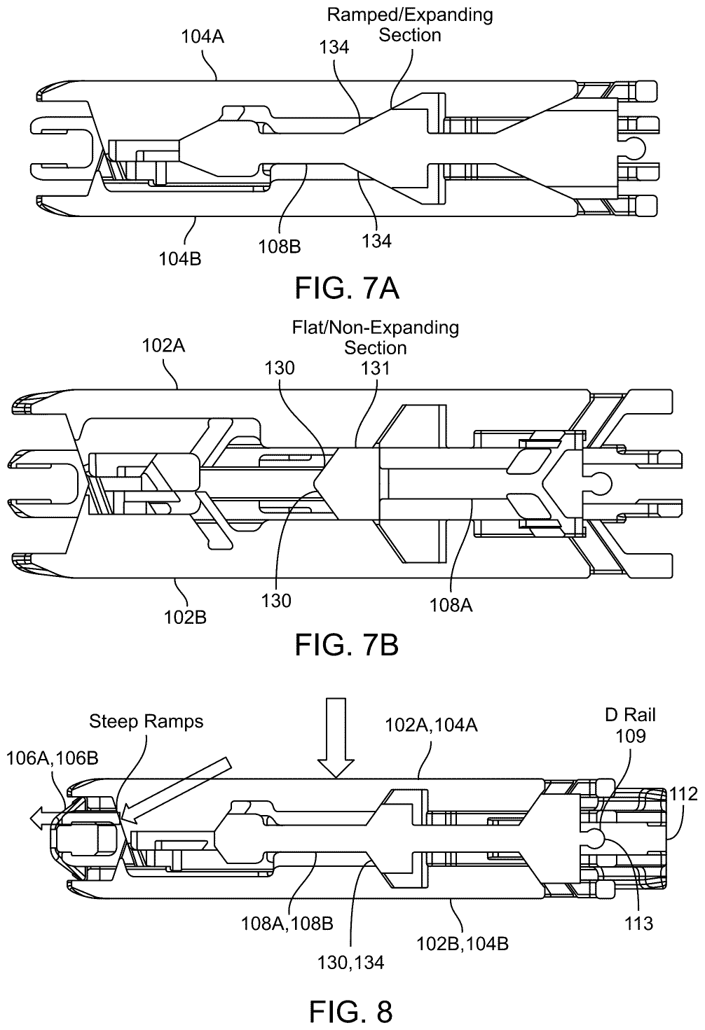

FIGS. 7A, 7B side views showing vertical expansion of the expandable spacer 100. The views show that unequal expansion between the anterior and posterior endplates is achieved by expanding only one set of the endplates while not expanding the other set. This is done using the section of the ramp which is flat for a particular distance, so when one set of endplates are in the flat section after expanding, the other set of endplates continue to expand.

FIG. 7A shows the posterior vertical expansion wedge 108B vertically expanding the posterior endplates 104A, 104B with the shallow ramp 134. FIG. 7B shows the anterior vertical expansion wedge 108A has completed vertical expansion of the anterior endplates 102A, 102B with the steep ramps and the anterior endplates 102A, 102B are now on the flat/non-expanding section 131 while the posterior endplates 104A, 104B continue to expand.

In use, the drive screw 116 is rotated in a first direction which translates the frame 114 toward the drive housing 112. During translation, the frame 112 contacts the “steep ramps” of lateral expansion wedges 106A, 106B causing them to translate laterally. As this lateral translation occurs, the vertical expansion wedges 108A, 108B contact the anterior endplates 102A, 102B and the posterior endplates 104A, 104B. The steep ramps 130 of the anterior expansion wedge 108A contacts and expands the anterior endplates 102A, 102B, and the shallow ramps 134 of the posterior expansion wedge 108B contacts and expands the posterior endplates 104A, 104B.

FIG. 8 shows the expandable spacer 100 collapsing. The drive screw 15 is rotated counter-clockwise with an instrument (not shown) which translates the frame 114 away from the drive housing 112 which contacts the “steep ramps” of lateral expansion wedges 106A, 106B causing them to translate inward. As the frame 114 translates away from the drive housing 112. the “d-rails” hold the vertical expansion wedges 108A, 108B stationary causing the anterior and posterior endplates 102, 104 to translate down the ramps 130, 134 of the vertical expansion wedges 108 and collapse.

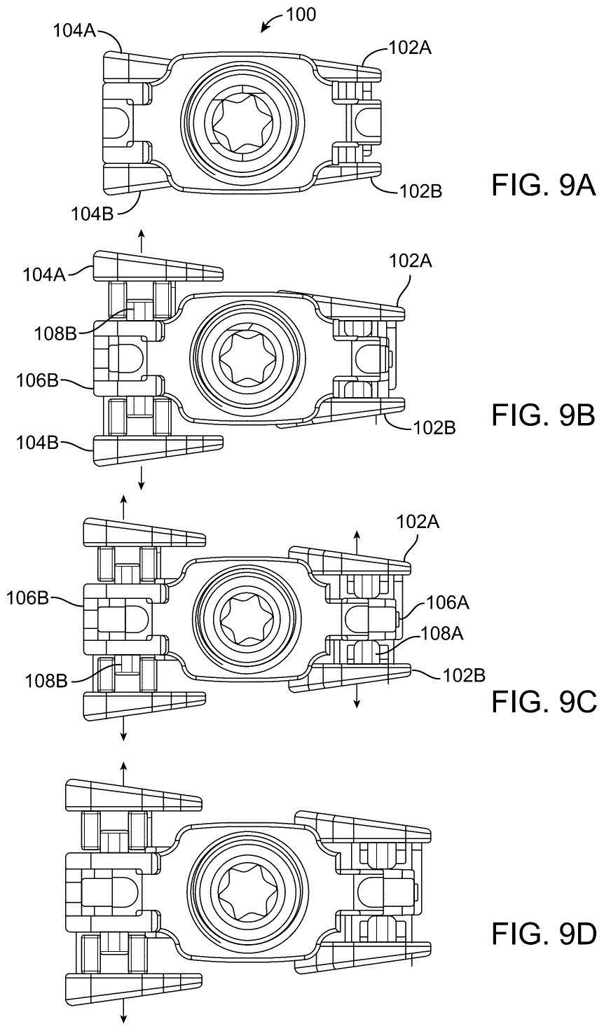

FIGS. 9A-9D show expansion of the expandable spacer 100, showing the expansion of the upper and lower anterior endplates 102A, 102B and the upper and lower posterior endplates 104A, 104B are at unequal rates. FIG. 9A shows the expandable spacer 100 in a collapsed or delivery configuration. As the drive screw 116 is rotated, the frame 114 translates toward the drive housing 112 and the shallow ramps 134 of posterior vertical expansion wedge 108B contact the upper and lower posterior endplates 104A, 104B and starts vertical expansion, shown in FIG. 9B. As the frame 114 continues translating toward the drive housing 112, the upper and lower posterior endplates 104A, 104B continue vertical expansion, and now the steep ramps of the anterior vertical expansion wedge 108A contact the upper and lower anterior endplates 102A, 102B and starts vertical expansion, so both sets of endplates are expanding, show in FIG. 9C. Once the upper and lower anterior endplates 102A, 102B engage the flat section 131 they stop expanding while the upper and lower posterior endplates 104A, 104B continue vertical expansion, shown in FIG. 9D. Once the upper and lower posterior endplates 104A, 104B reach the top of the shallow ramp, they will be fully expanded.

In the collapsed or delivery configuration, the expandable interbody spacer 100 has a delivery height and first width. When the screw 116 is rotated in a first direction, the frame 114 and drive housing 112 move toward each other and the upper and lower anterior endplates 102A, 102B and the upper and lower posterior endplates 104A, 104B vertically expand at unequal rates. The expandable interbody spacer 100 does not have to be completely expanded and can be stopped anywhere between collapsed state or the fully expanded state, depending on the expansion needed between the adjacent vertebrae.

In the expanded state the expandable interbody spacer 100 includes a central opening that may be filled with materials, such as bone graft, allograft, Demineralized Bone Matrix (“DBM”) or other suitable materials. To insert the materials, the graft insertion window 105 is sized to allow materials to be introduced into the central opening of expandable interbody spacer 100 once is place in desired position.

The upper and lower end plates 102, 104 may include surface features or treatment configured to promote bone growth that engage the bone. For example, the surface may be a textured surface or roughened surface to promote bone integration or the surface may use a coating or be chemically etched to form a porous or roughened surface. In some embodiments the surface may include teeth. Each of the upper and lower end plates may use the same surface feature or different surface feature.

The expandable interbody spacer 100 components may be fabricated from any biocompatible material suitable for implantation in the human spine, such as metal including, but not limited to, titanium and its alloys, stainless steel, surgical grade plastics, plastic composites, ceramics, bone, or other suitable materials. In some embodiments, surfaces on the components may be formed of a porous material that participates in the growth of bone with the adjacent vertebral bodies. In some embodiments, the components may include a roughened surface that is coated with a porous material, such as a titanium coating, or the material is chemically etched to form pores that participate in the growth of bone with the adjacent vertebra. In some embodiments, only portions of the components be formed of a porous material, coated with a porous material, or chemically etched to form a porous surface, such as the upper and lower surfaces that contact the adjacent vertebra are roughened or porous.

The expandable interbody spacer 100 may also be used with various tools, such as inserter tools, deployment tools and/or removal tools. The tools may include various attachment features to enable percutaneous insertion of the expandable interbody spacer 100 into the patient. For example, the tools may include arms or clamps to attach to the cutouts or other openings, slots or trenches of the drive mechanism. The tools may also include an actuation device to couple with the proximal section of the screw. Once the expandable interbody spacer 100 has been inserted and positioned within the intervertebral space between two vertebrae with the insertion tool, the deployment tool may actuate to deploy and expand the expandable interbody spacer 100 by applying a rotational force to screw.

In operation, the expandable interbody spacer 100 may be inserted into the intervertebral disc space between two vertebrae using an insertion tool. The insertion tool or a deployment tool may engage with the proximal end of the expandable interbody spacer 100. As the deployment tool applies the rotational force to the screw, the expandable interbody spacer 100 gradually expands as described above. In some cases, the expandable interbody spacer 100 may need to be removed with a removal tool.

Example embodiments of the methods and systems of the present invention have been described herein. As noted elsewhere, these example embodiments have been described for illustrative purposes only and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments but should be defined only in accordance with the following claims and their equivalents.

1. An expandable spacer with lordosis for placement between adjacent vertebrae comprising:
upper and lower anterior endplates having a first set of ramps;
an anterior vertical expansion wedge having steep ramps configured to engage the first set of ramps to vertically expand the upper and lower anterior endplates at a first expansion rate;
upper and lower posterior endplates having a second set of ramps; and
a posterior vertical expansion wedge having shallow ramps configured to engage the second set of ramps configured to vertically expand the upper and lower posterior endplates at a second expansion rate;
wherein the first expansion rate is not the same as the second expansion rate, the unequal expansion creates lordosis for the expandable spacer.

2. The expandable spacer of claim 1, further including a drive means coupled to the anterior and posterior expansion wedges.

3. The expandable spacer of claim 2, wherein the drive means includes a drive housing and a drive frame rotatably coupled to a drive screw configured to translate the drive housing and drive frame toward each other.

4. The expandable spacer of claim 3, further including anterior and posterior lateral expansion wedges coupled to the drive housing and drive frame, wherein actuation of the drive screw translates the drive housing toward the drive frame and laterally expand the anterior and posterior lateral expansion wedges away from each other.

5. The expandable spacer of claim 4, wherein the anterior and posterior vertical expansion wedges are positioned within anterior and posterior lateral expansion wedge slots.

6. An expandable spacer with lordosis for placement between adjacent vertebrae comprising:
anterior and posterior endplates having anterior and posterior vertical endplate ramps;
an anterior vertical expansion wedge with anterior vertical expansion wedge ramps configured to engage the anterior vertical endplate ramps;
a posterior vertical expansion wedge having posterior vertical expansion wedge ramps configured to engage the posterior vertical endplate ramps; and
a drive means coupled to the anterior and posterior expansion wedges, wherein actuation of the drive means is configured to translate anterior and posterior vertical expansion wedges and vertically expand the anterior endplates away from each other at a first expansion rate, and vertically expand the posterior endplates away from each other at the at a second expansion rate,
wherein the difference between the first and second expansion rates create lordosis for the expandable spacer.

7. The expandable spacer of claim 6, wherein the drive means includes a drive housing coupled to the anterior and posterior vertical expansion wedges, a drive frame, and a drive screw, the drive screw having a distal end rotatably coupled to the drive housing and a proximal end rotatably coupled to the drive frame, where actuation of the drive screw translates the drive housing toward the drive frame.

8. The expandable spacer of claim 7, further comprising anterior and posterior lateral expansion wedges having lateral expansion wedge ramps configured to engage lateral drive frame ramps and lateral drive housing ramps, wherein rotation of the drive screw translates the drive frame toward the drive housing and laterally translating the anterior and posterior lateral expansion wedges away from each other.

9. An expandable spacer with lordosis for placement between adjacent vertebrae comprising:
upper and lower anterior endplates having anterior endplate ramps;
an anterior expansion wedge having anterior expansion wedge ramps configured to engage the upper and lower anterior endplate ramps to vertically expand upper and lower anterior endplates;
upper and lower posterior endplates having posterior endplate ramps;
a posterior expansion wedge having posterior expansion wedge ramps configured to engage the upper and lower posterior endplate ramps to vertically expand the upper and lower posterior endplates; and
a drive means couple to the anterior and posterior expansion wedges, wherein activation of the drive means is configured to vertically expand the upper and lower anterior endplates away from each other at a first expansion rate and vertically expand the upper and lower posterior endplates away from each other at a second expansion rate, the difference between the first expansion rate and the second expansion rate creates lordosis for the expandable spacer.

10. The expandable spacer of claim 9, further comprising anterior and posterior lateral expansion wedges coupled to the drive means, wherein activation of the drive means is configured to laterally expand the anterior and posterior lateral expansion wedges away from each other.
